(19) United States
(12) Patent Application Publication
(10) Pub. No.: US 2025/0161071 A1
(43) Pub. Date: May 22, 2025

(54) INTERVERTEBRAL INSERT

(71) Applicant: SPINE DOCTORS CO., LTD., Daegu (KR)

(72) Inventor: Gun CHOI, Pohang-si, Gyeongsangbuk-do (KR)

(21) Appl. No.: 18/729,828

(22) PCT Filed: Feb. 4, 2022
(86) PCT No.: PCT/KR2022/001778

§ 371 Date: Jul. 17, 2024

(30) Foreign Application Priority Data
Jan. 18, 2022 (KR) 10-2022-0007227

(51) Int. Cl. A61F 2/46; A61F 2/28; A61F 2/30; A61F 2/44

(52) U.S. Cl. CPC A61F 2/4611; A61F 2/442; A61F 2/4465; A61F 2002/2835; A61F 2002/30354; A61F 2002/30579; A61F 2002/30789; A61F 2002/4627; A61F 2002/4629

(57) ABSTRACT

An intervertebral insert includes a lower edge formed in a rounded shape, thereby facilitating rotation, the lower edge entering an interbody space first to come in contact with the inside of the interbody space, and can be inserted with only one side of a vertebral joint removed, thereby reducing bleeding and increasing a patient's satisfaction.

### TECHNICAL FIELD

The present invention relates to an intervertebral insert, and more specifically, to an intervertebral insert, which initially enters between the vertebrae, has a gently formed lower edge in contact with the interior of the vertebral interbody to facilitate rotation, and which can be inserted after removing the vertebral joint of only one side, thus preserving the vertebral joint, reducing muscle damage or bleeding, and increasing patient satisfaction.

### BACKGROUND ART

The intervertebral disc is a cartilaginous structure that connects the vertebrae in the spine, enabling movement between two vertebral bodies and stably supporting and protecting the vertebral bodies. The intervertebral disc is also known as a disc.

The intervertebral disc consists of an annulus fibrosus and a nucleus pulposus. The annulus fibrosus regulates the movement of vertebral fragments, and the nucleus pulposus consists of 70 to 80% water. The intervertebral disc absorbs and transfers loads and shocks applied in the vertical direction. In degenerative disc disease, the annulus fibrosus weakens in movement or ability to contain the nucleus, and is reduced in water content. As a complex result, diseases such as spinal stenosis, osteophyte formation, disc herniation, and nerve root compression occur.

One of methods for treating diseases associated with the intervertebral disc is lumbar interbody fusion, typically performed by inserting a prosthetic material between the vertebral bodies. The prosthetic material is generally inserted to fuse adjacent vertebral bones after removing a damaged vertebra, and is manufactured in various shapes and sizes, such as a circular ring or a cuboid. Additionally, the prosthetic material may contain bone fragments harvested from a patient or other persons, or artificial bone fragments therein. In addition, the prosthetic material may have a hollow structure to enhance the fusion effect between the vertebral bodies. The prosthetic material is known by various terms, such as a cage, a spine insert, an artificial disc, and an implant.

Conventional posterior lumbar interbody fusion is a method of incising muscles of both vertebrae to secure an insertion space for inserting a spine insert, removing vertebral joints, and inserting two spine inserts. So, the conventional posterior lumbar interbody fusion has the problem of significant bleeding and slow patient recovery due to the incision sites. Moreover, anterior lumbar interbody fusion involves a high risk since a spine insert must enter from the front of the spine, where major structures such as the aorta and inferior vena cava are located. Additionally, lateral lumbar interbody fusion allows insertion of a cage suitable for the disc size, but hardly allows for decompression of the posterior nerves and has a risk of lumbar plexus injury and damage to the psoas muscle. Therefore, the lumbar interbody fusion needs a spine insert technology to achieve objects, such as reducing blood loss, shortening surgery time, and decreasing patient morbidity.

As a conventional art, Korean Patent Publication No. 10-2021-0087342 “spinal surgery cage” is described.

### DISCLOSURE

### Technical Problem

Accordingly, the present invention has been made in view of the above-mentioned problems occurring in the related art, and it is an object of the present invention to provide an intervertebral insert, which initially enters between the vertebrae, has a gently formed lower edge in contact with the interior of the vertebral interbody to facilitate rotation, and which can be inserted after removing the vertebral joint of only one side, thus preserving the vertebral joint, reducing muscle damage or bleeding, and increasing patient satisfaction.

### Technical Solution

To accomplish the above-mentioned objects, according to the present invention, there is provided an intervertebral insert including: an insertion part which is initially inserted into a space between vertebral bones, which is the vertebral interbody, and formed to be gradually narrower towards the insertion direction to facilitate insertion; a body part which extends rearward from the insertion part, and has a height to support and maintain the vertebral interbody; a bone graft filling hole which is formed to penetrate the upper surface and the lower surface so that bone graft material is filled; a bone graft transfer hole which is formed to be penetrated at both sides, and is communicated with the bone graft filling hole so that the filled bone graft material can fill the space between the vertebral bodies compactly; and a coupling part which extends rearward from the body part, and is formed to be coupled with the intervertebral insertion mechanism used in lumbar interbody fusion.

Moreover, the intervertebral insert forms a lower edge extending to the insertion part and the body part, and the lower edge is gently formed to facilitate rotation within the vertebral interbody.

Furthermore, the body part includes: a plurality of protrusions which are formed to protrude towards the vertebral bones on both sides, and are arranged at regular intervals continuously, each of which has a consistent incline at the front and rear sides, wherein the rear incline is more inclined than the front incline to facilitate the entry of the intervertebral insert and prevent the backward movement.

Additionally, the coupling part includes: a coupling hole which is formed in the center of the rear end surface, and has an internal thread; and a pair of fixing grooves which are formed at a predetermined distance above and below from the coupling hole, and are formed to have a predetermined depth inward.

In addition, an intervertebral insert according to a second embodiment of the present invention further includes a pair of expansion parts which are formed to extend vertically from the body part to secure a support area inside the vertebral interbody.

Moreover, the body part includes: an accommodation hole which is formed to penetrate the upper and lower surfaces of the body part between the plurality of bone graft filling holes, has a locking groove formed on the inner circumferential surface thereof to accommodate the expansion part therein; and an insertion groove which is formed toward the insertion part from the position of the bone graft transfer hole facing the coupling hole, is communicated with the accommodation hole, and is formed to have a predetermined depth on the inner circumferential surface of the accommodation hole.

Furthermore, the expansion part includes: an expansion bar which is accommodated in the accommodation hole and moves vertically; an insertion rod which is formed to be attachable and detachable to the intervertebral insert insertion mechanism, and moves the expansion bar outward from the body part while being inserted into the insertion groove; and an expansion head part which is connected to the expansion bar, is formed to protrude from the upper and lower surfaces of the body part, and has a filling protrusion formed to have a length in the back-and-forth direction and inserted into the bone graft filling hole on the surface which is in contact with the body part.

Additionally, the expansion bar is accommodated in the accommodation hole, has a protrusion and a spring formed on the outer side thereof, and moves along the locking groove in the vertical direction, and an end of the expansion bar towards the center of the body part is curved.

In addition, when the insertion rod is inserted into the insertion groove, one end of the insertion rod touches the curve of the expansion bar, presses the expansion bar, and advances toward the insertion part, so that the expansion bar moves outward from the body part.

Furthermore, multiple insertion rods with different thicknesses are provided, such that a thickness that allows the expansion of the expansion part appropriately for a surgical site is selected, and coupled to the intervertebral insert insertion mechanism.

### Advantageous Effect

According to embodiments of the present invention, the intervertebral insert can be inserted by removing the vertebral joint of one side, thereby reducing muscle damage and bleeding and increasing patient satisfaction.

Moreover, the intervertebral insert of the present invention can be formed substantially larger than the conventional intervertebral insert, thereby enabling the formation of a solid fusion.

Furthermore, the intervertebral insert of the present invention can easily rotate due to the lower edge having a gradual slope.

Additionally, the intervertebral insert of the present invention can secure a wider support area inside the vertebral interbody through the expansion part.

In addition, the intervertebral insert of the present invention includes insertion rods of various thicknesses, thereby adjusting the expansion part according to surgical sites.

The effects according to the embodiments of the present invention mentioned above are not limited to the described content only, but may also include all effects that can be expected from the specification and drawings.

### DESCRIPTION OF DRAWINGS

FIG. 1 is a perspective view of an intervertebral insert according to a first embodiment of the present invention.

FIG. 2 is a side view of FIG. 1.

FIG. 3 is a side cross-sectional view of FIG. 1.

FIG. 4 is a rear view of FIG. 1.

FIG. 5 is a perspective view of an intervertebral insertion mechanism for inserting the intervertebral insert according to the first embodiment of the present invention.

FIG. 6 is a cross-sectional view of FIG. 5.

FIG. 7 is a perspective view illustrating a rod to which the intervertebral insert of FIG. 1 and the intervertebral insertion mechanism of FIG. 5 are combined.

FIGS. 8A and 8B are exemplary views illustrating a process of inserting the intervertebral insert of FIG. 1 between vertebral bodies.

FIG. 9 is an exemplary view illustrating that the intervertebral insert of FIG. 1 is adjusted in position by a intervertebral insert rotation mechanism.

FIG. 10 is a perspective view of an intervertebral insert according to a second embodiment of the present invention.

FIG. 11 is an exploded perspective view of FIG. 10.

FIG. 12 is a side cross-sectional view of FIG. 10.

FIG. 13 is a perspective view illustrating a state in which an insertion rod is coupled to an intervertebral insertion mechanism for inserting the intervertebral insert according to the second embodiment of the present invention.

FIGS. 14A and 14B are exemplary views illustrating the operation of the intervertebral insert of FIG. 10.

FIGS. 15A to 15C are exemplary views illustrating a process of inserting the intervertebral insert of FIG. 10 between vertebral bodies.

FIGS. 16A and 16B are exemplary views illustrating the intervertebral insert of FIG. 10 varying according to the thickness of the insertion rods.

### MODE FOR INVENTION

Hereinafter, the description of the present invention, with reference to the attached drawings is not limited to specific embodiments, and can undergo various modifications and have various embodiments. Moreover, the contents described below should be understood to include all transformations, equivalents, or substitutes falling within the spirit and scope of the present invention.

The terms “first,” “second,” etc., used in the description below are used to describe various components, not intended to limit the components but solely used to distinguish one component from another.

The same reference numbers used throughout the specification designate the same components.

The singular form used in the present invention may be understood into the plural form unless otherwise specifically stated in the context. It should be also understood that the terms of ‘include’ or ‘have’ in the specification are used to mean that there are characteristics, numbers, steps, operations, components, parts, or combinations of the steps, operations, components and parts described in the specification and there is no intent to exclude existence or possibility of other characteristics, numbers, steps, operations, components, parts, or combinations of the steps, operations, components and parts.

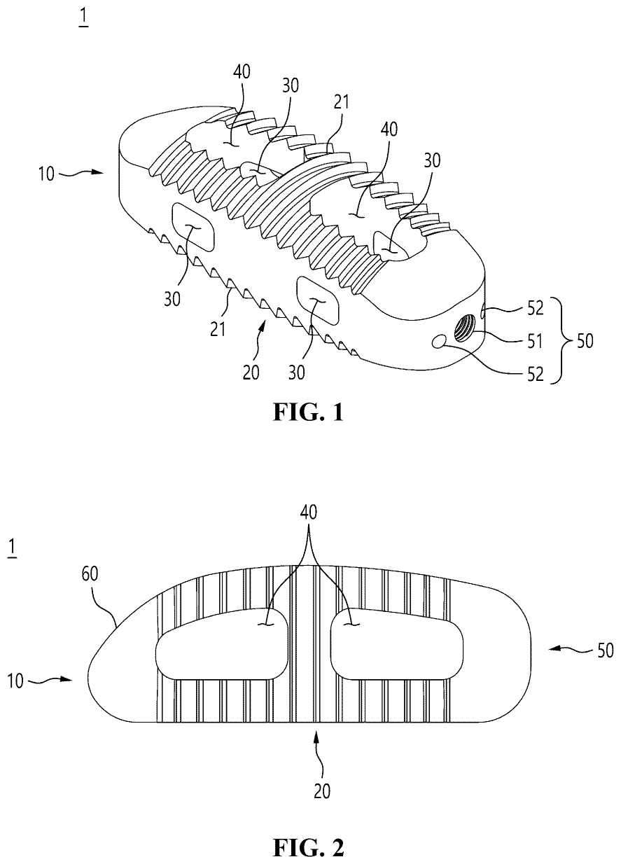

Hereinafter, the embodiments of the present invention will be described in detail with reference to FIGS. 1 to 16.

Additionally, in the context of the intervertebral insert being inserted between vertebral bodies, the direction of the intervertebral insert inserted between vertebral bodies is referred to as “forward”, and the opposite direction to the forward direction is referred to as “rearward.” In addition, terms indicating directions such as “left”, “right”, “upward”, and “downward” are defined to indicate each direction based on the aforementioned forward and rearward directions.

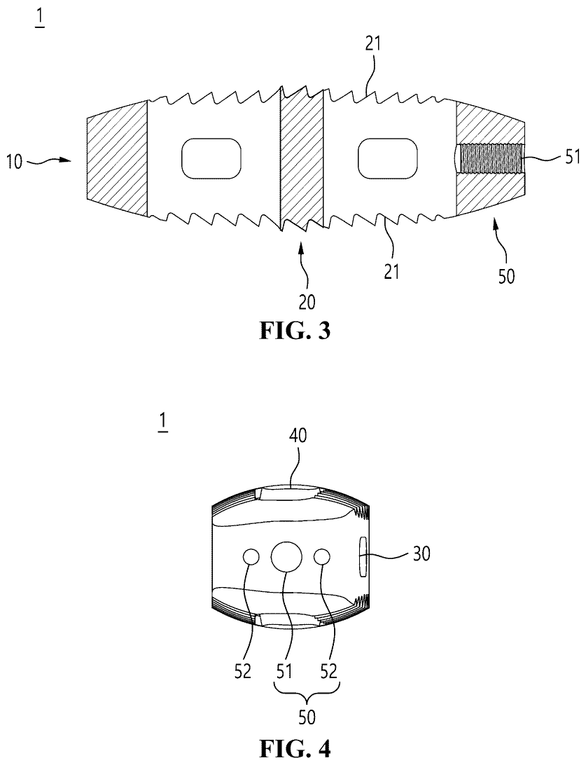

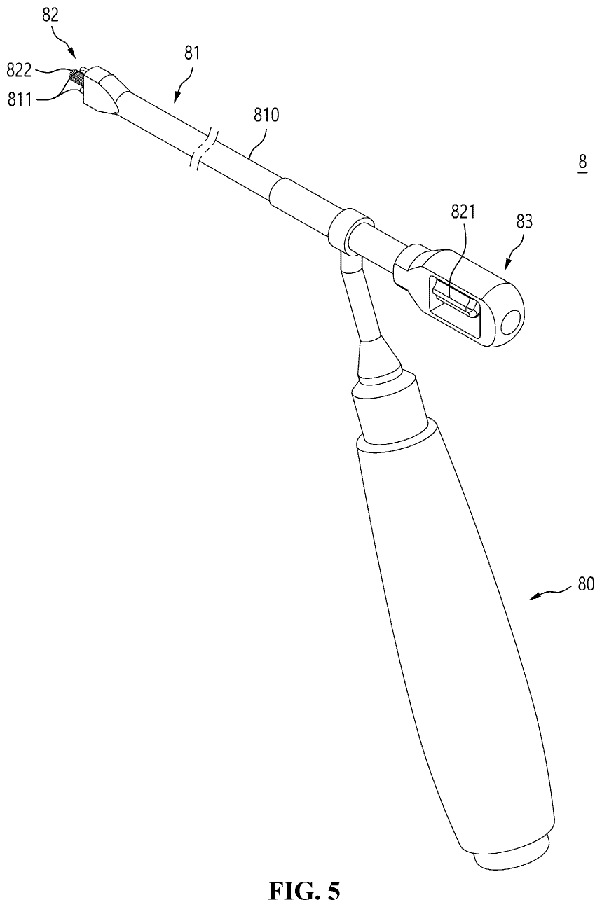

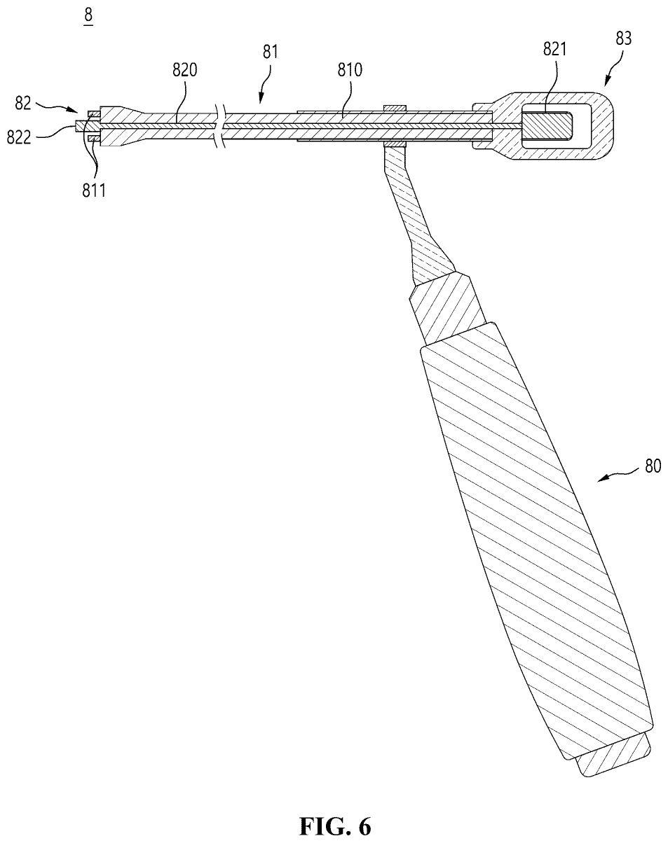

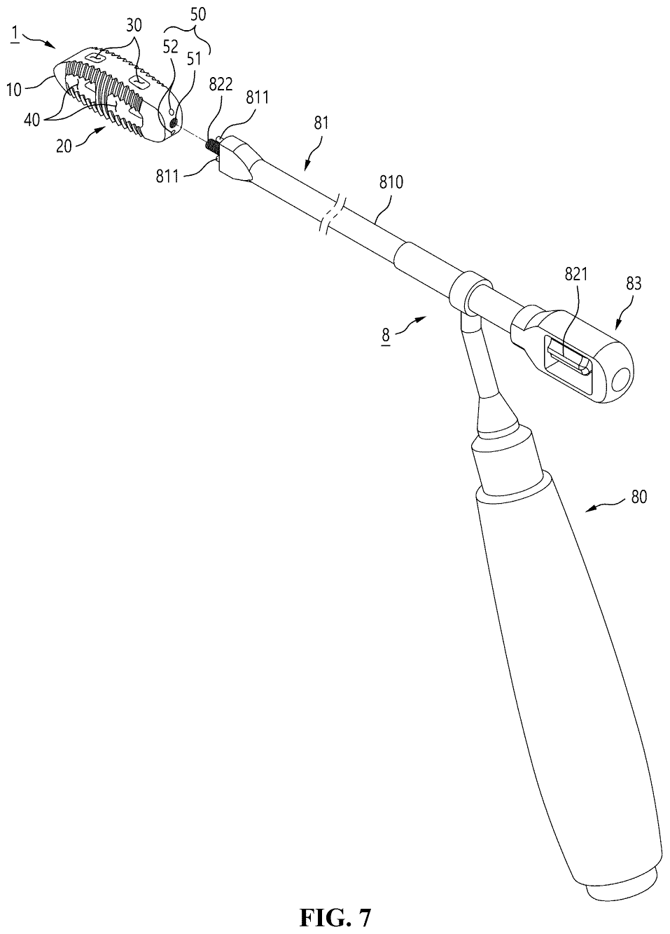

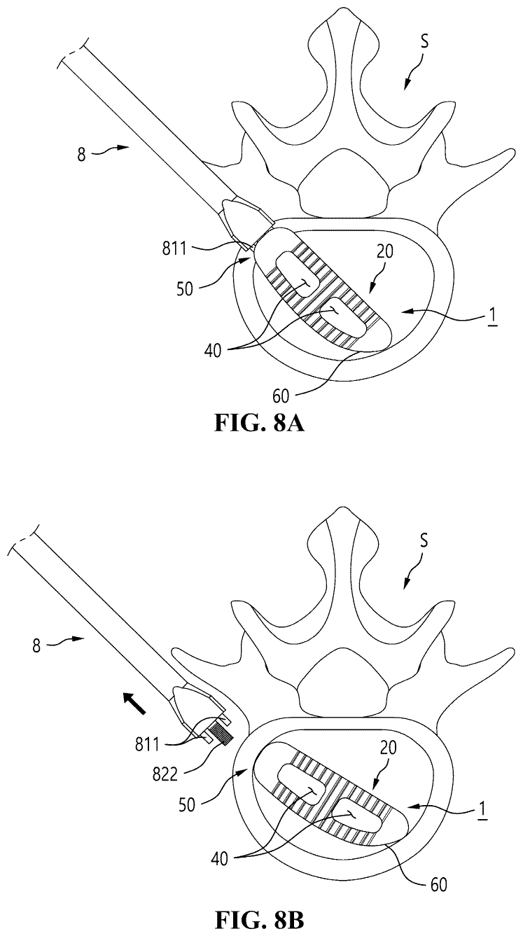

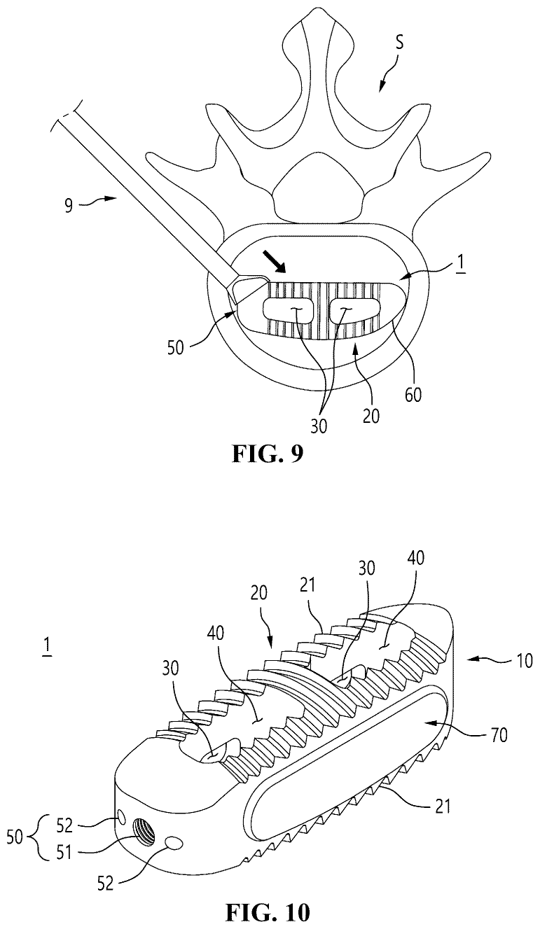

FIG. 1 is a perspective view of an intervertebral insert according to a first embodiment of the present invention, FIG. 2 is a side view of FIG. 1, FIG. 3 is a side cross-sectional view of FIG. 1, FIG. 4 is a rear view of FIG. 1, FIG. 5 is a perspective view of an intervertebral insertion mechanism for inserting the intervertebral insert according to the first embodiment of the present invention, FIG. 6 is a cross-sectional view of FIG. 5, FIG. 7 is a perspective view illustrating a rod to which the intervertebral insert of FIG. 1 and the intervertebral insertion mechanism of FIG. 5 are combined, FIGS. 8a and 8b are exemplary views illustrating a process of inserting the intervertebral insert of FIG. 1 between vertebral bodies, and FIG. 9 is an exemplary view illustrating that the intervertebral insert of FIG. 1 is adjusted in position by a intervertebral insert rotation mechanism.

First, in the technical field of the present invention, an intervertebral insert 1 is used in lumbar interbody fusion. After an intervertebral disc positioned between damaged vertebral bones of the human body, the intervertebral insert 1 is inserted into the vertebral interbody, which is a space between two adjacent vertebral bones S to maintain the original height of the intervertebral disc and restore the function of the vertebrae.

The lumbar interbody fusion is classified according to the insertion direction of the intervertebral insert 1 into posterior lumbar interbody fusion (PLIF), transforaminal lumbar interbody fusion (TLIF), extraforaminal lumbar interbody fusion (EFLIF, ELIF), oblique lateral lumbar interbody fusion (OLIF), and anterior lumbar interbody fusion (ALIF). The intervertebral insert 1 according to the embodiments of the present invention is preferably used for extraforaminal lumbar interbody fusion (EFLIF, ELIF), but is not limited thereto, and can be used in all the above-mentioned lumbar interbody fusion methods.

Specifically, the extraforaminal lumbar interbody fusion (EFLIF, ELIF) is the surgery of removing vertebral joints through the Kambin's triangle working zone and inserting the intervertebral insert 1 using an intervertebral insertion mechanism 8. Here, Kambin's triangle working zone refers to the space formed among the lateral edge of the traversing nerve, the nerve root, and the upper endplate of the lower vertebra. The present invention relates to an intervertebral insert 1 used in lumbar interbody fusion, particularly, in extraforaminal lumbar interbody fusion (EFLIF, ELIF).

Referring to FIGS. 1 to 9, the intervertebral insert 1 according to the first embodiment of the present invention may include an insertion part 10, a body part 20, a bone graft filling hole 30, a bone graft transfer hole 40, and a coupling part 50.

The insertion part 10 is initially inserted into the space between the vertebral bones S, which is the vertebral interbody, and may have a shape gradually narrower towards the insertion direction to facilitate insertion.

The body part 20 extends rearward from the insertion part 10, and may have height and size which can support and maintain the vertebral interbody.

Furthermore, the body part 20 may have a plurality of protrusions 21. The plurality of protrusions 21 are formed to protrude towards the vertebral bones S on both sides, and may be arranged at regular intervals continuously. It is preferable that the protrusion 21 has a consistent incline at the front and rear sides of the protrusion, wherein the rear incline is more inclined than the front incline to facilitate the entry of the intervertebral insert 1 and prevent the backward movement, but the present invention is not limited thereto. Additionally, the protrusion 21 can ensure more robust fusion with the two vertebral bones without sliding within the vertebral interbody.

The bone graft filling hole 30 is formed to be penetrated at the upper surface and the lower surface so that bone graft material can be filled. The bone graft transfer hole 40 is formed to be penetrated at both sides, and communicates with the bone graft filling hole 30 so that the filled bone graft material can fill the space between the vertebral bodies compactly.

The bone graft filling hole 30 and the bone graft transfer hole 40 can ensure the uninterrupted filling of the bone graft material inside the vertebral interbody and increase the density of the filled bone graft material to prevent creating empty spaces.

Here, the bone graft material can be filled into the bone graft filling hole 30 and the bone graft transfer hole 40 after the intervertebral insert 1 has been inserted between the vertebral bodies. The bone graft material filled in the bone graft filling hole 30 and the bone graft transfer hole 40 can facilitate bone growth over time, enabling fusion between the vertebral bodies.

The bone graft material may be autograft bone pieces or osteoinductive proteins harvested from the patient's iliac crest, but is not limited thereto, and all types of bone graft materials used in the field of the present invention are applicable.

The coupling part 50 extends rearward from the body part 20, and can be formed to be coupled with the intervertebral insertion mechanism 8 used in the lumbar interbody fusion. For this purpose, the coupling part 50 may include a coupling hole 51 and a pair of fixing grooves 52.

The coupling hole 51 is formed in the center of the rear end surface, and may have an internal thread to be coupled with a coupling protrusion 822 of the intervertebral insertion mechanism 8.

The pair of fixing grooves 52 are formed at a predetermined distance above and below from the coupling hole 51, has a predetermined depth inward, such that a fixing protrusion 811 of the intervertebral insertion mechanism 8 can be inserted into the fixing grooves.

Additionally, the intervertebral insert 1 may form a lower edge 60 extending to the insertion part 10 and the body part 20. The lower edge 60 is a portion which first enters the vertebral interbody and is in contact with the interior of the vertebral interbody, and preferably, has a gradual slope to facilitate rotation within the vertebral interbody, but is not limited thereto.

In addition, the intervertebral insert 1 can be manufactured from inert rigid metals, rhodium, platinum, medical-grade stainless steel, cobalt-chromium-molybdenum alloy, zirconium oxide, zirconium, titanium, plastic, hafnium, PEEK, or the likes, and most preferably, manufactured from PEEK material which has excellent machinability and durability. However, the intervertebral insert is not limited thereto, and any biocompatible material which is harmless to a body can be used.

Moreover, the intervertebral insert 1 can accommodate a marker (not shown) in the longitudinal direction, allowing medical personnel to adjust the position of the marker while checking the position of the marker during surgery. Here, any type of marker used in the field of the present invention can be used, and the position and the manner in which the marker is accommodated within the intervertebral insert 1 are not limited.

Referring to FIGS. 5 to 7, the intervertebral insertion mechanism 8 for inserting the intervertebral insert 1 into the vertebral interbody according to the first embodiment of the present invention may include a handle part 80, a fixing part 81, a rotation part 82, and a bracket part 83.

The handle part 80 can be gripped by hand to move the intervertebral insertion mechanism 8 so that the intervertebral insert I can be inserted into the vertebral interbody. After the insertion of the intervertebral insert 1, the handle part 80 can be formed to minimize the movement for proceeding to the next step. For this purpose, it is preferable for the handle part 80 to be formed in a vertical direction to be gripped without bending the hand, but is not limited thereto.

The fixing part 81 can include a fixing body 810 and a pair of fixing protrusions 811.

The fixing body 810 is formed in a tubular shape to accommodate the rotation part 82 inside. The pair of fixing protrusions 811 are formed at a predetermined distance from the hole at the front end of the fixing body 810, and have a predetermined length outward to be inserted into the fixing groove 52 of the intervertebral insert 1.

The rotation part 82 can be accommodated inside the fixing body 810 to be rotatable. For this purpose, the rotation part 82 can include a rotation body 820, a rotation handle 821, and a coupling protrusion 822.

The rotation body 820 is accommodated inside the fixing body 810, is formed in a bar shape with a circular cross-section to be rotatable, and can be formed longer than the fixing body 810.

The rotation handle 821 is formed at the rear end of the rotation body 820, has a diameter larger than that of the rotation body 820, and includes protrusions formed along the outer circumference to facilitate rotation with less force and allow for easy rotational control.

The coupling protrusion 822 is formed at the front end of the rotation body 820 to protrude from the fixing body 810, and has a thread, which is formed on the outer circumferential surface thereof to be screw-coupled with the thread of the coupling hole 51 of the intervertebral insert 1.

The bracket part 83 is formed at the rear side of the fixing part 81 and rotation part 82, is formed to couple with the outer circumference of the other side of the fixing body 810, and has a hole formed in the back-and-forth direction to accommodate the rotation body 820. Additionally, the bracket part 83 has a lateral hole formed to expose the rotation handle 821, making it easier for medical personnel to operate the rotation handle 821.

As illustrated in FIG. 7, the intervertebral insert I can be coupled with the intervertebral insertion mechanism 8. Specifically, the intervertebral insert 1 can be coupled by inserting the fixing protrusions 811 of the intervertebral insertion mechanism 8 into the fixing grooves 52, and by manipulation of the rotation handle 821, the rotation body 820 and the coupling protrusion 822 can rotate and are screw-coupled into the coupling hole 51. In this instance, medical personnel can manipulate the rotation handle 821 with one hand while holding the handle part 80 in the other hand, enabling precise operations.

Referring to FIGS. 8a and 8b, the intervertebral insert 1 can be inserted into the vertebral interbody while being coupled to the intervertebral insertion mechanism 8 outside the human body. Subsequently, by operating the rotation handle 821 of the intervertebral insertion mechanism 8, the coupling protrusion 822 can be separated from the coupling hole 51 and the fixing protrusions 811 can be separated from the fixing grooves 52. Thus, the intervertebral insert 1 remains inside the vertebral interbody and the intervertebral insertion mechanism 8 can be removed from the human body.

Meanwhile, in general, the intervertebral insert 1 is inserted and rotated using the intervertebral insertion mechanism 8. However, in the state in which the intervertebral insert 1 is coupled with the intervertebral insertion mechanism 8, when the intervertebral insertion mechanism 8 receives a blow from a medical hammer, the screw-coupling between the intervertebral insert 1 and the intervertebral insertion mechanism 8 can be easily loosened, and the thread of the coupling protrusion 822 may get damaged.

Referring to FIG. 9, the intervertebral insert 1 inserted into the vertebral interbody can be rotated using an intervertebral insert rotation mechanism 9. The intervertebral insert rotation mechanism 9 is formed with an end that has a predetermined surface area to increase contact area with the intervertebral insert 1.

Here, the intervertebral insert rotation mechanism 9 can be in contact with the upper edge of the inclined intervertebral insert 1, and force can be transferred from the upper side to the lower side of the intervertebral insert 1 using a medical hammer or a similar tool. Consequently, the intervertebral insert 1 can receive force from the intervertebral insert rotation mechanism 9, and rotate to adjust the position thereof within the vertebral interbody.

Thus, the intervertebral insert 1 of the present invention can be rotated and adjusted in position within the vertebral interbody using the intervertebral insert rotation mechanism 9. Additionally, as the intervertebral insertion mechanism 8 only performs the role of inserting the intervertebral insert 1, thereby preventing damage.

Furthermore, the intervertebral insert 1 can be easily rotated when being rotated by a blow of the intervertebral insert rotation mechanism 9 since the lower edge 60, which first enters and contacts the interior of the vertebral interbody, has a gentle slope.

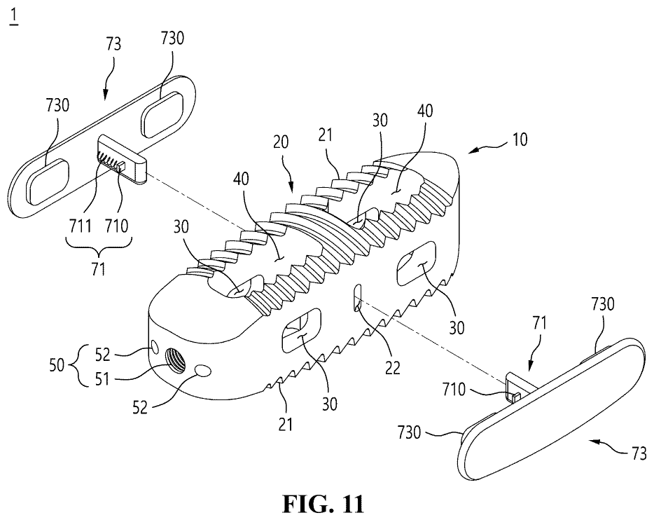

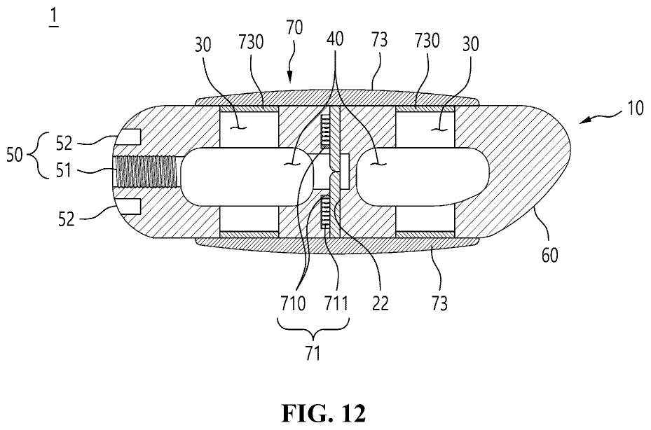

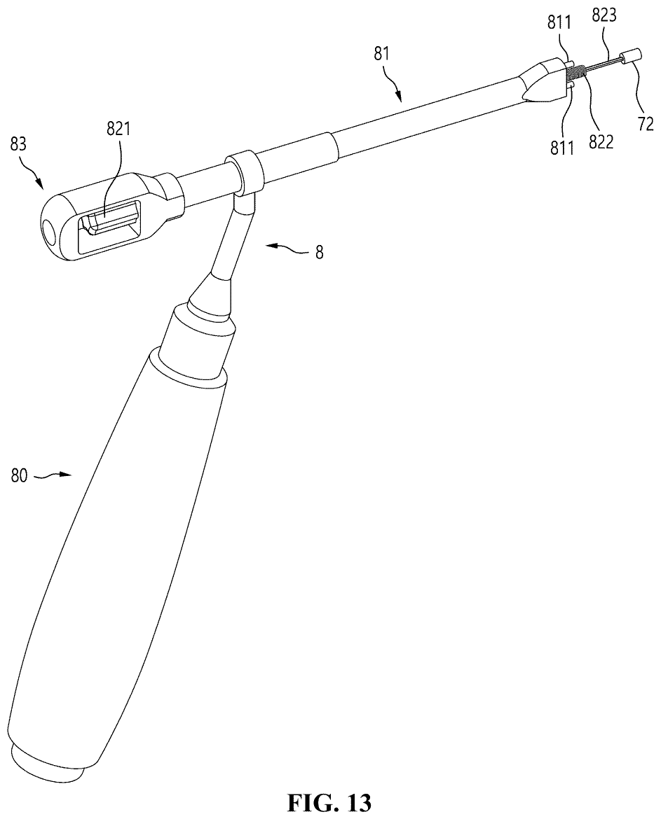

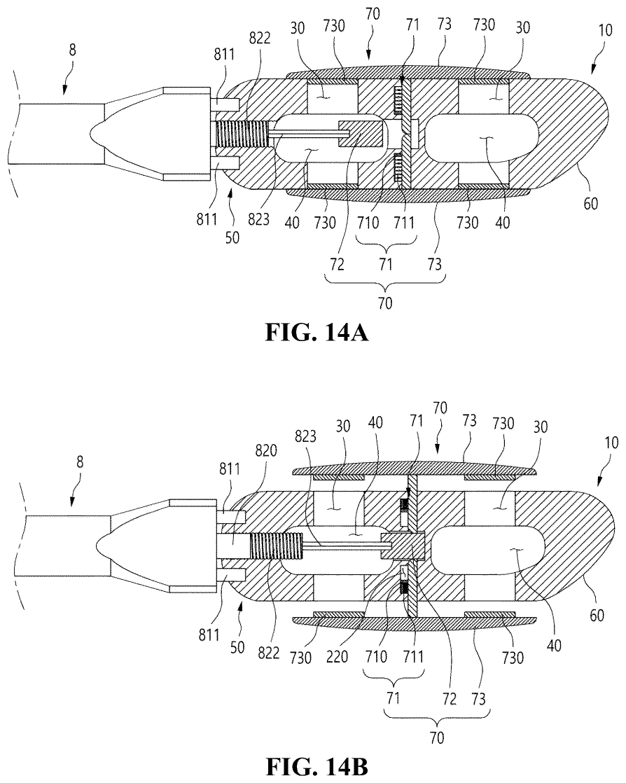

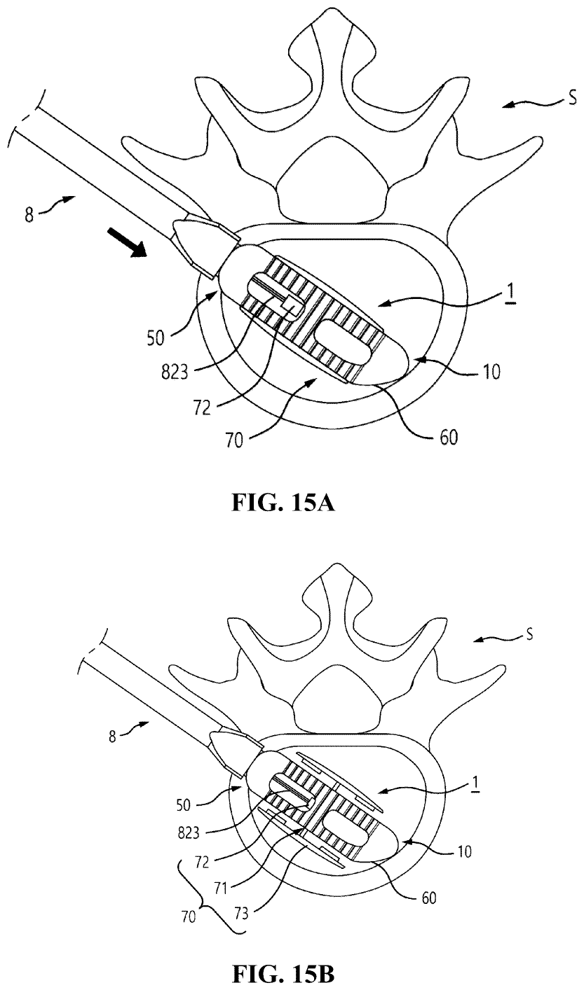

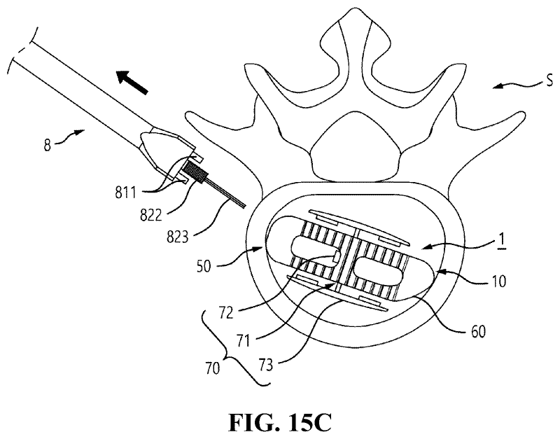

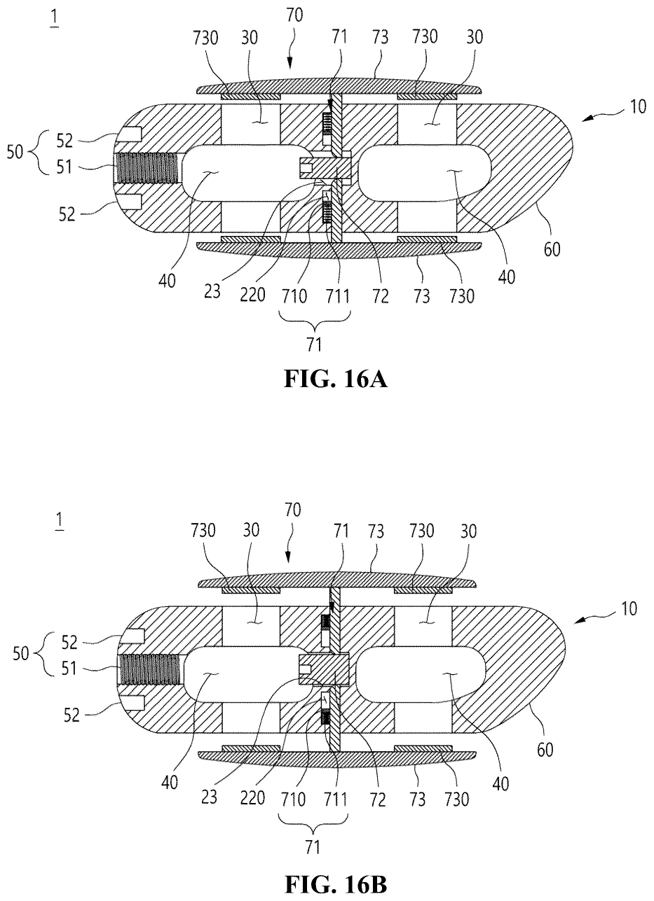

FIG. 10 is a perspective view of an intervertebral insert according to a second embodiment of the present invention, FIG. 11 is a exploded perspective view of FIG. 10, FIG. 12 is a side cross-sectional view of FIG. 10, FIG. 13 is a perspective view illustrating a state in which an insertion rod is coupled to an intervertebral insertion mechanism for inserting the intervertebral insert according to the second embodiment of the present invention, FIGS. 14A and 14B are exemplary views illustrating the operation of the intervertebral insert of FIG. 10, FIGS. 15A to 15C are exemplary views illustrating a process of inserting the intervertebral insert of FIG. 10 between vertebral bodies, and FIGS. 16A and 16B are exemplary views illustrating the intervertebral insert of FIG. 10 varying according to the thickness of the insertion rods.

Referring to FIGS. 10 to 16B, the intervertebral insert 1 according to the second embodiment of the present invention may further include a pair of expansion parts 70.

Here, except the expansion parts 70 and the body part 20, the intervertebral insert 1 according to the second embodiment of the present invention can be substantially identical to the intervertebral insert 1 according to the first embodiment of the present invention described above.

Therefore, only the expansion parts 70 and the body part 20 will be described in detail.

Before describing the expansion parts 70, the configuration of the body part 20 which enables the operation of the expansion part 70 will be described.

The body part 20 may include an accommodation hole 22 and an insertion groove 23.

The accommodation hole 22 is a space where the expansion part 70 is accommodated, and can be formed to be penetrated at the upper and lower surfaces of the body part 20 between the plurality of bone graft filling holes 30. Additionally, the accommodation hole 22 has a locking groove 220 formed on the inner surface thereof to prevent the separation of the expansion part 70.

The insertion groove 23 is formed toward the insertion part 10 from the position of the bone graft transfer hole 40 facing the coupling hole 51, can communicate with the accommodation hole 22, and may be formed to have a predetermined depth on the inner circumferential surface of the accommodation hole 22.

The pair of expansion parts 70 may be formed to extend vertically from the body part 20, and can be configured to secure a support area inside the vertebral interbody. For this purpose, the expansion part 70 can include an expansion bar 71, an insertion rod 72, and an expansion head part 73.

The expansion bar 71 is accommodated in the accommodation hole 22, has a protrusion 710 and a spring 711 formed on the outer side thereof, moving along the locking groove 220 in the vertical direction. Additionally, an end of the expansion bar 71 towards the center of the body part 20 can be curved.

The insertion rod 72 is formed to be attachable and detachable to the intervertebral insertion mechanism 8, and can move the expansion bar 71 outward from the body part 20 while being inserted into the insertion groove 23. The insertion rod 72 preferably has a smaller diameter than the diameter of the coupling hole 51 and the coupling protrusion 822 to penetrate through the coupling hole 51 of the body part 20, but is not limited thereto.

The expansion head part 73 is connected to the expansion bar 71 and is formed to protrude from the upper and lower surfaces of the body part 20, and can be formed to have a length in the back-and-forth direction. The expansion head part 73 can have a filling protrusion 730 to facilitate the support of the vertebral interbody by the expansion part 70. The filling protrusion 730 can be formed on the surface which is in contact with the body part 20 and inserted into the bone graft filling hole 30.

In addition, it is preferable for the expansion head part 73 to be formed at the same height as the body part 20 to support the vertebral interbody, but is not limited thereto. Preferably, the thickness of the expansion head 73 protruding vertically should be formed so as not to hinder the insertion of the intervertebral insert 1 into the vertebral interbody.

In this instance, the intervertebral insertion mechanism 8 for inserting the intervertebral insert 1 into the vertebral interbody may further include an extension bar 823.

The extension bar 823 is formed in a bar shape having a specific length, of which the front end is coupled with the insertion rod 72 and the rear end is coupled with the coupling protrusion 822. The extension bar 823 may have a diameter smaller than those of the insertion rod 72 and the coupling protrusion 822. The cross-section of the extension bar 823 may be circular, hexagonal, etc., and the insertion rod 72 and the coupling protrusion 822 can be formed to be couplable with the extension bar. All types of coupling methods applicable to the field of the present invention can be used.

Referring to FIGS. 14A and 14B, the expansion part 70 of the intervertebral insert 1 can be operated using the intervertebral insertion mechanism 8.

Specifically, as illustrated in FIG. 14A, the intervertebral insert I can be coupled with the intervertebral insertion mechanism 8 where the extension bar 823 and the insertion rod 72 are coupled with each other. Thereafter, as illustrated in FIG. 14B, when the insertion rod 72 is inserted into the insertion groove 23, one end of the insertion rod 72 touches the curve of the expansion bar 71, presses the expansion bar 71, and advances toward the insertion part 10, so that the expansion bar 71 moves outward from the body part 20.

Referring to FIGS. 15A to 15C, the intervertebral insert 1 can be inserted into the vertebral interbody from outside the human body while being coupled to the intervertebral insertion mechanism 8. In this instance, the extension bar 823 is coupled to the front surface of the coupling protrusion 822 of the intervertebral insertion mechanism 8, and the insertion rod 72 is coupled to the front surface of the extension bar 823. Next, as illustrated in FIG. 15B, when the rotation handle 821 of the intervertebral insertion mechanism 8 is manipulated, the insertion rod 72 can be inserted into the insertion groove 23 while advancing toward the insertion part 10.

Finally, when the rotation handle 821 of the intervertebral insertion mechanism 8 is manipulated in the opposite direction, the insertion rod 72 and the extension bar 823 can be separated from each other, the coupling protrusion 822 can be separated from the coupling hole 51, and the fixing protrusion 811 can be separated from the fixing groove 52. Thus, the intervertebral insert 1 remains inside the vertebral interbody and the intervertebral insertion mechanism 8 can be removed from the human body.

Moreover, multiple insertion rods 72 with different thicknesses can be provided. The insertion rod 72 of a thickness that allows the expansion of the expansion part 70 appropriately for a surgical site can be selected, and coupled to the intervertebral insertion mechanism 8 for use. So, medical personnel can pre-calculate the support area in which the expansion part 70 is formed suitable for a patient's body and select one of the multiple insertion rods 72.

Specifically, as illustrated in FIG. 16A, since the insertion rod 72 of the intervertebral insert 1 can be inserted and coupled into the insertion groove 23 inside the body part 20, the expansion head 73 can expand vertically. On the other hand, in case of a patient with a larger intervertebral disc size, as illustrated in FIG. 16B, an insertion rod 72 which is larger than the insertion rod 72 used in FIG. 16A can be used to allow the expansion head 73 to expand further outward. Thus, the intervertebral insert 1 can secure a wider support area inside the vertebral interbody to be suitable for the patient's body.

The embodiments of the present invention have been described with reference to the attached drawings. However, it will be understood by those skilled in the art that the disclosure may be embodied in other concrete forms without changing the technological scope and essential features. Therefore, the above-described embodiments should be considered only as examples in all aspects and not for purposes of limitation.

### SEQUENCE LIST TEXT

1: intervertebral insert
10: insertion part
20: body part
21: protrusion part
22: accommodation hole
220: locking groove
23: insertion groove
30: bone graft filling hole
40: bone graft transfer hole
50: coupling part
51: coupling hole
52: fixing groove
60: lower edge
70: expansion part
71: expansion bar
711: spring
72: insertion rod
73: expansion head part
730: filling protrusion
8: intervertebral insert insertion mechanism
80: handle part
81: fixing part
810: fixing body
1811: fixing protrusion
82: rotation part
820: rotation body
821: rotation handle
822: coupling protrusion
823: extension bar
83: bracket part
9: intervertebral insert rotation mechanism
S: vertebral bone

1-10. (canceled)

11. An intervertebral insert comprising:
an insertion part configured to be initially inserted into a vertebral interbody and has a shape gradually narrower towards an insertion direction to facilitate insertion;
a body part extending from the insertion part in a direction opposite to the insertion direction, the body part having a height to support and maintain the vertebral interbody;
a bone graft filling hole configured to be penetrated at an upper surface and a lower surface of the body part to be filled with bone graft material;
a bone graft transfer hole configured to be penetrated at both sides of the intervertebral insert, and is configured to communicate with the bone graft filling hole to fill the vertebral interbody with the bone graft material compactly; and
a coupling part extending from the body part in the direction opposite to the insertion direction, the coupling part being configured to be coupled with a intervertebral insertion mechanism used in lumbar interbody fusion.

12. The intervertebral insert according to claim 11, wherein the intervertebral insert forms a lower edge extending from the insertion part to the body part, and the lower edge has a predetermined slope to facilitate rotation within the vertebral interbody.

13. The intervertebral insert according to claim 11, wherein the body part includes:
a plurality of protrusions configured to protrude towards vertebral bones from the both sides of the intervertebral insert, the plurality of protrusions being disposed at regular intervals continuously,
wherein each of the plurality of protrusions has a first incline at a front side of each of the plurality of protrusions and a second incline at a rear side of each of the plurality of protrusions, and
wherein the second incline is more inclined than the first incline to facilitate an entry of the intervertebral insert and prevent a backward movement of the intervertebral insert.

14. The intervertebral insert according to claim 11, wherein the coupling part includes:
a coupling hole defined at a center of a rear end surface of the intervertebral insert, the coupling hole having an internal thread; and
a pair of fixing grooves
wherein one of the pair of fixing grooves is defined at a predetermined distance above the coupling hole along a height direction of the body part, and the other of the pair of fixing grooves is defined at a predetermined distance below the coupling hole along the height direction of the body part,
wherein each of the pair of fixing grooves has a predetermined depth.

15. The intervertebral insert according to claim 14, wherein the intervertebral insert further includes a pair of expansion parts extending in the height direction from the body part to provide a support area inside the vertebral interbody.

16. The intervertebral insert according to claim 15,
wherein the bone graft filling hole has a plurality of bone graft filling holes, and
wherein the body part includes:
an accommodation hole configured to be penetrated at the upper and lower surfaces of the body part between the plurality of bone graft filling holes,
wherein the accommodation hole has a locking groove defined on an inner circumferential surface of the accommodation hole to accommodate therein each of the pair of expansion parts; and
an insertion groove defined toward the insertion part from a position of the bone graft transfer hole facing the coupling hole,
wherein the insertion groove is configured to communicate with the accommodation hole, and has a predetermined depth on the inner circumferential surface of the accommodation hole.

17. The intervertebral insert according to claim 16, wherein each of the pair of expansion parts includes:
an expansion bar configured to be accommodated in the accommodation hole and to move along the height direction;
an insertion rod attachable and detachable to the intervertebral insertion mechanism, the insertion rod being configured to move the expansion bar outward from the body part while being inserted into the insertion groove; and
an expansion head part connected to the expansion bar,
wherein the expansion head part protrudes from the upper or the lower surface of the body part, and
wherein the expansion head part has a filling protrusion having a length in the insertion direction, the filling protrusion being configured to be inserted into the bone graft filling hole.

18. The intervertebral insert according to claim 17,
wherein the expansion bar is configured to be accommodated in the accommodation hole, has a protrusion of the expansion bar and a spring disposed on an outer side of the expansion bar, and configured to move along the locking groove in the height direction,
wherein a distal end of the expansion bar has a curve.

19. The intervertebral insert according to claim 18, wherein when the insertion rod is inserted into the insertion groove, one end of the insertion rod is configured to touch the curve of the expansion bar, to press the expansion bar at the curve, and to advance toward the insertion part, thereby moving the expansion bar outward from the body part.

20. The intervertebral insert according to claim 17,
wherein the insertion rod has a plurality of insertion rods,
wherein each of the plurality of insertion rods has a thickness different from a thickness of another of the plurality of insertion rods.
wherein each of the plurality of insertion rods is configured to be coupled to the intervertebral insertion mechanism interchangeably to allow different levels of expansion of the pair of expansion parts for a surgical site selected.
